(19) United States
(12) Patent Application Publication
(10) Pub. No.: US 2013/0171192 A1
(43) Pub. Date: Jul. 4, 2013

(54) ADJUVANT FORMULATIONS FOR BACTERIAL AND VIRUS VACCINES AND METHOD OF MAKING SAME

(71) Applicant: IRX Therapeutics, Inc., New York, NY (US)

(72) Inventors: John W. Hadden, Cold Spring Harbor, NY (US); Paul H. Naylor, Hazel Park, MI (US); Kathy L. Signorelli-Petrat, Springfield, VA (US)

(73) Assignee: IRX Therapeutics, Inc., New York, NY (US)

(21) Appl. No.: 13/658,518

(22) Filed: Oct. 23, 2012

Related U.S. Application Data
(63) Continuation of application No. 10/500,602, filed on Feb. 22, 2005, now abandoned.
(60) Provisional application No. 60/308,139, filed on Jul. 27, 2001.

(51) Int. Cl. A61K 39/39

(52) U.S. Cl. CPC A61K 39/39
USPC 424/208.1; 424/184.1; 424/204.1; 424/234.1; 424/265.1; 424/246.1; 424/211.1

(57) ABSTRACT

A pharmaceutical composition including an adjuvant effective amount of a protected inosine monophosphate (IMP) compound. The pharmaceutical composition includes the protected IMP compound alone, or in combination with vaccine agents with or without additional adjuvants. The pharmaceutical composition can be utilized as a vaccine composition or can be included with existing vaccine compositions in order to increase a specific T lymphocyte mediated immune response thereto. Various methods relating to the pharmaceutical composition and the vaccine are described herein. The vaccines can be employed to prevent or treat infections. Additionally, the pharmaceutical compositions not only increase T-cell responses, but also confer, by pretreatment, non-specific protection against a variety of pathogens. This combination of actions is appropriate for enhancing defense against bioterrorism with organisms like smallpox and anthrax.

### RELATED APPLICATIONS

This application claims the benefit under 35 U.S.C. §120 of U.S. application Ser. No. 10/500,602, entitled “ADJUVANT FORMULATIONS FOR BACTERIAL AND VIRUS VACCINES AND METHOD OF MAKING SAME” filed on Feb. 22, 2005, which is herein incorporated by reference in its entirety, which is a national stage application of Application PCT/US2002/023765, which in turn claims priority under 35 U.S.C. §119(e) to U.S. Provisional Application Ser. No. 60/308,139, entitled “ADJUVANT FOR BACTERIAL AND VIRUS VACCINES AND METHOD OF MAKING SAME” filed on Jul. 27, 2001, which is herein incorporated by reference in its entirety.

### BACKGROUND OF THE INVENTION

1. Field of the Invention

The present invention relates to pharmaceutical compositions. More specifically, the present invention relates to adjuvant formulations for vaccines with enhanced T-cell directed immunogenic activity and with the additional capacity to protect a host from pathogen challenge during the immunization process (immunoprophylaxis).

2. Description of Related Art

According to the World Health Organization, twenty-five percent of worldwide mortality is the result of infectious diseases. Viral and bacterial infections are a major public health concern. In both of these categories, bacterial and viral pathogens avoid the host immune system by growing within the host's cells. Cellular immunity, mediated by T lymphocytes and by macrophages that are activated by specific T lymphocyte products, is the general mode of host defense against most pathogens, while antibody response confers against helminths and some viruses, particularly hepatitis A viruses.

Everyone is susceptible to developing infectious diseases. Regardless of ago or health status, exposure to pathogens and subsequent development of infectious disease occurs. Certain populations, however, such as the elderly and those who are immunocompromised can be more susceptible to developing infectious disease. As a result, exposure to bacterial or viral pathogens can lead to mortality.

Elderly individuals and those individuals who are immunocompromised exhibit changes in their immune system that make them more susceptible to viral diseases and less responsive to vaccines. Much of the decrease in immunoresponsiveness is associated with changes in the T-cell compartment. B-cells and associated antibody responses are less affected. One of the most consistent changes noted with advancing age is the decrease in the proportion of naïve T-cells with a concomitant increase in those with a “memory” phenotype. Hence, fewer cells are available to respond to the new antigens present in a vaccine. In addition, the T-cells of aged individuals show a decline in cellular proliferation and changes in cytokine responses. Specifically, there is a decrease in the frequency of T-cells that produce the critical cytokine IL-2 and a decrease in the number of receptors for this cytokine. Thus, lowered production of and capacity to respond to IL-2 is a common component of immunosenescence and characterizes a shift from Th1 to Th2 dominance.

Despite the problems associated with elderly and immunocompromised individuals, vaccination still remains the best source of protection against pathogens, particularly viruses. Vaccines have been used as an effective form of treatment and/or prevention of infectious diseases. To date, vaccines have had a tremendous impact on world health. Currently, modern vaccines, both prophylactic and therapeutic, have been and are being developed against critical and potentially devastating infectious diseases. Often though, vaccines do not confer fully protective immunity. Poorly immunogenic viral antigens, a lack of time between vaccination and exposure, and the inability of the vaccinated individual to respond are some reasons. Moreover, some viruses or bacteria develop resistance to treatment regimes.

In addition to not generating an immune response of sufficient magnitude, some vaccines are very slow in developing immunity against diseases such as anthrax, hepatitis B infections, and the like. Further, many injections are needed over time so agents that speed the process are needed. Also, many vaccines have difficulties in stimulating an appropriate type of response best tailored to combating a given infection or disease. While current vaccines typically generate adequate humoral responses, there is an increasing need for activating cell-mediated immunity (“CMI”) to fight obligate intracellular pathogens, particularly chronic viral infections such as HIV and HCV. For instance, some vaccine therapeutic strategies would require stimulation of T-cell specific responses. Thus, there is a need for compounds that enhance not only the overall magnitude of the immune response, but also foster the development of cell-mediated Th1 response. The majority of vaccines described to date have demonstrated the ability to enhance antibody responses while very few are known to have specific T-cell and/or cell-mediated immunity stimulating effects.

Treatment with substances possessing immunopotentiating properties is one way of increasing an individual's immune system speed and effectiveness toward infectious diseases. In fighting these diseases, the presence of sensitized T-lymphocytes and activated macrophages are key factors in immunity. Therefore, effective treatments must activate macrophages and sensitize T-lymphocytes (Ryan, Stites and Terr, 637-645; and Mills, Stites and Terr, 646-656).

Substances that stimulate or enhance cellular immunity in the context of vaccination are especially important for those individuals with conditions resulting in immunosuppression. These situations include infection with HIV, treatment with immunosuppressive drugs, or the presence of certain metabolic conditions such as diabetes mellitus and renal failure, and aging since loss of T-cell specific immune function occurs with aging. In such immunocompromised patients, the ability to develop immunity in response to vaccination is reduced, so that despite vaccination, such individuals remain at risk for serious consequences of infectious disease.

One substance used to improve an immune response is an adjuvant. Generally, an adjuvant is an agent that acts via multiple mechanisms to increase an immune response to a particular vaccine antigen, thus increasing efficacy and potentially reducing the quantity of antigen necessary in the vaccine to generate protective responses. Adjuvants work in a variety of manners. For instance, adjuvants can directly affect T-cells. T-cell adjuvanticity is the intentional use of a compound with an immunogen to elicit preferentially or exclusively a T-cell response as measured by the generation of cytotoxic T-cells or delayed-type hypersensitivity (“DTH”). Based on the literature, two areas of discussion are germane to the development of exclusively a T-cell response. First, cytokines play a pivotal role in modulating T-cell responses. Second, specific components can enhance T-cell growth, differentiation, and activation. Cytokines are critical for both the magnitude and the focus of the immune response.

Recent evidence has implicated specific receptors on antigen presenting cells (“APC's”), especially the dendritic cells, as targets for many currently known adjuvants. These receptors recognize ligands found in bacteria that serve to activate the innate immune response. Designated as TLR (toll-like receptors), they are activated by agents such as flagella, lipopolysaccharide (“LPS”), lipid A, lipoproteins, double-stranded RNA, and bacterial-like stretches such as CpG. These ligands activate a myriad of intracellular proteins and appear to enhance uptake of antigen, maturation, and movement to the lymph node and expression of co-stimulatory proteins of the dendritic cells. For example, a recent study using oligonucleotide microarrays demonstrated that whole pathogens activated both common and pathogen-specific genes.

Adjuvants have various other uses including, but not limited to: increasing the amount of antibody and number of effector T-cells produced; reducing the quantity of antigen needed; reducing the time to protection; reducing frequency of treatment; retaining the antigen locally near the site of administration to produce a depot effect facilitating a slow, sustained release of antigen to cells of the immune system; promoting and attracting accumulation of immunoreactive cells at the site of injection and/or the antigen depot; stimulating immunoreactive cells to elicit immune responses; maintaining serum antibody levels; modifying activities of cells that are concerned with generating and maintaining the immune response; modifying the presentation of antigen to the immune system; activating host macrophages, dendritic cells, B-cells and T-cells; increasing antigen uptake; up-regulating antigen processing; stimulating cytokine release; stimulating B-cell switching and maturation; and eliminating immunosuppressive cells.

Desirable characteristics of adjuvants include: (1) lack of toxicity; (2) ability to stimulate a long-lasting immune response; (3) simplicity of manufacture and stability in long-term storage; (4) ability to elicit both CMI and humoral immune response (“HIR”) to antigens administered by various routes, if required; (5) synergy with other adjuvants; (6) capability of selectively interacting with populations of antigen presenting cells (“APC”); (7) ability to specifically elicit appropriate Th1 or Th2 cell-specific immune responses; and (8) ability to selectively increase appropriate antibody isotype levels (e.g., IgA) against antigens.

There are various categories and types of adjuvants. For instance, there are intrinsic adjuvants, such as lipopolysaccarides, which are normally the components of the killed or attenuated bacteria used as vaccines. Additionally, there are extrinsic adjuvants, which are immunomodulators that are typically noncovalently mixed with antigens and are formulated to enhance the host immune response. A wide range of extrinsic adjuvants can provoke potent immune responses to antigens. These include saponins complexed to membrane protein antigens, i.e., immune stimulating complexes, polymers with mineral oil, Freund's complete (FCA) or incomplete adjuvant (FIA), bacterial products such as muramyl dipeptide (“MDP”) and “LPS”, as well as lipid A, monophosphoryl lipid A, liposomes, proteoliposomes, and copolymers. To efficiently induce “HIR” and “CMI”, immunogens are often emulsified with the adjuvant in oil.

A number of aliphatic nitrogenous bases have also been proposed for use as immunologic adjuvants, including amines, quarternary ammonium compounds, guanidines, benzamidines and thiouroniums. Specific compounds include dimethyldioctadecylammonium bromide (DDA) and N,N-dioctadecyl-N,N-bis(2-hydroxyethyl)propanediamine (“avridine”). Aluminum hydroxide and aluminum phosphate (collectively referred to as alum) also are routinely used as adjuvants in human and veterinary vaccines.

Chemically defined adjuvants, such as monophosphoryl lipid A, phospholipid conjugates have been investigated (see Goodman-Snitkoff et al., J. Immunol. 147:410-415 (1991)) as has encapsulation of the protein within a proteoliposome (see Miller et al., J. Exp. Med. 176:1739-1744 (1992)). Synthetic polymers have also been evaluated as adjuvants. These include the homo- and copolymers of lactic and glycolic acids, which have been used to produce microspheres that encapsulate antigens (see Eldridge et al., Mol. Immunol. 28:287-294 (1993)).

Nonionic block copolymers are another synthetic adjuvant being evaluated. Adjuvant effects have also been investigated for low molecular weight copolymers in oil-based emulsions (see Hunter et al., The Theory and Practical Application of Adjuvants (Ed. Stewart-Tull, D. E. S.). John Wiley and Sons, N.Y., pp 51-94 (1995)) and for high molecular weight copolymers in aqueous formulations (Todd et al., Vaccine 15:564-570 (1997)).

As an alternative to chemical adjuvants, the use of cytokines as natural adjuvants has attracted some interest (Heath and Playfair, 1992). Various cytokines have been shown to be effective immunological adjuvants in a variety of model systems, enhancing protection induced by bacterial, viral and parasitic antigens. In the murine model, cytokines such as IL-1, IL-2, IFN-γ, and GM-CSF have been shown to have adjuvant effects for humoral, and to a lesser degree, cellular responses (Staruch and Wood, 1983; Nunberg et al., 1989; Playfair and deSouza, 1987). Limited adjuvanticity of TNF also has been reported by Ghiara, et al., 1987.

Other adjuvants are derived from bacteria. For example, the lipid-A portion of gram negative bacterial endotoxin and trehalose dimycolate of mycobacteria have been used as adjuvants. The phospholipid lysolecithin exhibited adjuvant activity (Arnold et al., Eur. J. Immunol. 9:363-366, 1979). Some synthetic surfactants exhibited adjuvant activity, including dimethyldioctadecyl ammonium bromide (DDA) and certain linear polyoxypropylenepolyoxyethylene (POP-POE) block polymers (Snippe et al., Int. Arch. Allergy Appl. Immunol. 65:390-398, 1981; and Hunter et al., J. Immunol. 127:1244-1250, 1981). While these natural or synthetic surfactants demonstrate some degree of adjuvant activity, they do not demonstrate the degree of immunopotentiation (i.e., adjuvant activity) as FCA or FIA.

Other adjuvants have been derived from a muramyl-peptide in the cell wall of bacteria. The smallest fragment of this molecule that retains adjuvant activity is N-acetyl-muramyl-L-alanyl-D-isoglutamine, which is also called muramyl dipeptide (“MDP”) (Ellouz et al., Biochem. & Biophys. Res. Comm. 1317-1325, 1974). There have been many MDP derivatives prepared as vaccine adjuvants and described in U.S. Pat. Nos. 4,158,052; 4,323,559; 4,220,637; 4,323,560; 4,409,209; 4,423,038; 4,185,089; 4,406,889; 4,082,735; 4,082,736; 4,427,659; 4,461,761; 4,314,998; 4,101,536; and 4,369,178, the disclosures of each of which are incorporated by reference herein.

In general, the above adjuvant agents and strategies augment humoral immunity more than cellular immunity. As a result, a lack of T cell adjuvants exists (Hadden, 1994). One novel area wherein adjuvant activity could be found is in agents that preferentially influence the development of the T-cell immune response at the cellular level. One such T-cell enhancing agent is methyl inosine monophosphate (“MIMP”). This agent and its analogs act as protected IMPs (U.S. Pat. No. 5,614,504) and stimulate T-lymphocyte differentiation and function and have the potential to move the immune response towards the cell-mediated immune response. A study showing that MIMP can enhance the cell-mediated immune response to sheep erythrocyte antigen has been reported (Sosa, et al., 1992). To date, however, the action of MIMP as a vaccine adjuvant has not been reported. Many adjuvants are antigen-specific and action to induce protection against one antigen does not necessarily predict protection against another.

MIMP has been shown to be effective agents for enhancing a variety of immune responses. As a result of the methyl substitution, MIMP is resistant to hydrolysis by 5′ nucleotidase and has shown efficacy as an oral formulation for human use.

In vitro experiments show that MIMP at concentrations of 1-100 μg/ml augments the response to PHA and Con A of lymphocytes from young and elderly human donors as well as from mice. Further in vitro studies found that MIMP induced T-cell markers in human bone marrow prothymocytes. Markers enhanced by short-term (2-4 hours) MIMP incubation were CD₃, IL-2 receptor and HLA-DR. Exposure to MIMP for six to eight hours results in the enhancement of CD₄ and CD₈ markers as well.

Using a classic Immune model, Sosa, et al. demonstrated that oral and parenteral MIMP administered at the time of immunization with sheep red blood cells (“SRBC”), results in a dose-dependent enhancement of the anti-SRBC antigen response. This enhancement is consistent with T-helper cell activation because SRBC response is T-cell dependent. Additional evidence for enhancement of T-cell immune activity is supported in studies demonstrating DTH response to SRBC challenge in the footpad. While these studies provided the basis for further experiments to demonstrate that MIMP is a T-cell adjuvant, they do not demonstrate efficacy as a T-cell adjuvant in a vaccine.

Another experiment has also been performed to assess the utility of MIMP as an oral vaccine adjuvant (U.S. Pat. No. 5,614,504). Certain mice strains are poor responders to the hepatitis B surface antigen (“HbsAg”) based vaccine (given as part of a Dane's particle type vaccine). Mice are given MIMP (50 mg/kg) orally 30 minutes prior to immunization or intraperitoneally simultaneously with immunization. One group also received oral MIMP for an additional 3 days after the simultaneous administration with immunization (MIMP daily group). Immunization is 16 mg of antigen per mouse in PBS at day 0 and 14. Antibody titer is assessed after primary immunization and booster immunization. While this study demonstrated an increase in antibody titer (B-cell response), it did not demonstrate that MIMP could enhance T-cell immune activity.

Various patents disclose the use of inosine-related compounds. U.S. Pat. No. 3,728,450 to Gordon discloses complexes formed by inosine and amino-alcohols, which have pharmacological activity in combating influenza or herpes virus. U.S. Pat. No. 4,221,794 to Hadden et al. discloses complexes of purine derivatives (9-(hydroxyalkyl) purines) with amino-alcohol salts of p-acetamidobenzoic acid, which have immunomodulating and antiviral activities. U.S. Pat. No. 4,221,909 to Hadden et al. discloses p-acetamidobenzoic acid salts of 9-(hydroxyalkyl) purines useful as viricides, immunoregulators and anti-leukemia agents. U.S. Pat. No. 4,340,726 to Giner-Sorolla et al. discloses esters (purine compounds) having immunomodulating, antiviral, anti-tumor and enzyme inhibitor activities. U.S. Pat. No. 4,221,910 to Giner-Sorolla et al. discloses 9-(hydroxyalkyl) purines useful as immunopotentiators, viricides and anti-leukemia agents. U.S. Pat. No. 4,457,919 to Giner-Sorolla et al. discloses purine derivatives that have immunomodulating, antiviral and anti-tumor activities. U.S. Pat. Nos. 4,510,144 and 4,387,226 to Giner-Sorolla et al. disclose dihydrothiazolo purine derivatives with immunomodulating activity.

U.S. Pat. No. 5,614,504 to Hadden, et al. discloses that protected IMP compounds resistant to hydrolysis by 5′ nucleotidase and having in vivo immunomodulating activity. The '504 patent specifically discloses MIMP, which, when tested in vivo in mice augmented T-cell dependent humoral responses, DTH sensitization responses, and mitogen responses. Previous publications on MIMP do not disclose its potential to selectively enhance T-cell mediated responses to a vaccine and did not envision that its capacity to protect against lethal microbial challenge can be coupled with its adjuvant activity to yield a combined defense against a variety of pathogens including those employed in bioterrorism.

Accordingly, there is a need for compositions and methods for increasing vaccine immunogenicity that can optimize T-cell responses and protection against lethal challenges.

### SUMMARY OF THE INVENTION

According to the present invention, there is provided a pharmaceutical composition including an adjuvant effective amount of a protected inosine 5′-monophosphate (“IMP”) compound. The pharmaceutical composition includes the protected IMP compound alone, or in combination with additional adjuvants. The present invention can be utilized as a vaccine composition or be included with existing vaccine compositions in order to increase immune responses thereof. The present invention also provides for various methods relating to the pharmaceutical composition and the vaccine described herein. These methods include, but are not limited to, various therapeutic uses and prophylaxis for protection of a subject. Specifically, the present invention optimizes T-cell responses and can be used to prevent or treat infection. The present invention confers nonspecific protection to a variety of pathogens by pretreatment. Thus, a combination of nonspecific protection with reduction of specific resistance increases likelihood of survival.

### DESCRIPTION OF THE DRAWINGS

Other advantages of the present invention will be readily appreciated as the same becomes better understood by reference to the following detailed description when considered in connection with the accompanying drawings wherein:

FIG. 1 is a schematic, chemical formulation of methyl inosine 5′-monophosphate (“MIMP”);

FIG. 2 is a bar graph illustrating in vitro MIMP treatment of human peripheral blood lymphocytes (“PBL”) exposed to an immunosuppressive HIV peptide, wherein normal PBL's are exposed to gp41 HIV peptide at 12.5 μM along with varying concentrations of MIMP, the solid line represents the control proliferation without the inhibitory peptide, and the results expressed as CPM+/−SEM and are representative of five donors tested with identical protocols;

FIG. 3 is a bar graph illustrating the effect of MIMP on anti-SRBC PFC response when given orally by gavage (PO) to eight-week-old female Balb/c mice, wherein MIMP is administered simultaneously with immunization of mice with 1×10⁶ SRBC, PFC are enumerated after four days, and results represent mean, and SEM from two experiments with eight to nine animals in each dose group shows a significant difference from a control (*<0.05);

FIG. 4 is a bar graph illustrating the effect of MIMP on 24-hour DHT response when given orally by gavage (PO) to eight-week-old Balb/c female mice on the day of immunization, wherein the mice are immunized with 1×10⁶ SRBC in the footpad, and the results represent mean, and SEM from two experiments with three to five animals in each dose group shows significant difference from control (*<0.05);

FIG. 5 is a chart illustrating the effect of influenza virus challenge infection on the survival of mice being administered MIMP by the intraperitoneal route, wherein MIMP is administered at either of two intervals related to influenza challenge (−7 days or −1 day; PBS at −1 day served as the control), both 100 μg (data shown) and 200 μg (data not shown) are effective, and the mean survival for the −1 day MIMP is 11.0+/−1.15 days compared to 9.2+/−1.03 days for the control (p<0.004);

FIG. 6 is bar graph illustrating the effect of MIMP on HBVSAg immunization, wherein mice are given MIMP (50 mg/kg) orally 30 minutes prior to immunization or simultaneously (intraperitoneal) with immunization, one group also received oral MIMP for an additional 3 days after simultaneous administration with immunization (MIMP daily group), immunization is 16 mg/mouse in PBS at days 0 and 14, and antibody titer is assessed after primary immunization (day 14) and booster immunization (day 14);

FIG. 7 is a bar chart illustrating that MIMP reverses IL-10 inhibition of PHA-induced proliferation of Human Lymphocytes in vitro, wherein Ficoll-Hypaque purified normal human peripheral blood lymphocytes (5×10⁵/ml) were cultured with 0.5 μg/ml PHA alone (open bar) or PHA with 5 ng/ml recombinant IL-10 (cross-hatched bars) that lead to suppression of proliferation as measured by *H-thymidine incorporation and co-incubation of MIMP (1-100 μg/ml) with PHA+IL-10 (10 ng/ml) overcame the suppressive effect (data is expressed as epm+S.E.M.);

FIG. 8 is a bar chart illustrating MIMP enhancement of the average footpad swelling for 5 immunized mice per group in response to subcutaneous immunization with flu antigen, wherein the doses of adjuvant for immunization are Identified on the X-axis of the bar chart;

FIG. 9 is a graphical illustration of antibody titer as average optical density in Flu antibody ELISA assays for dilutions of serum from groups of 5 mice immunized with flu antigen and the indicated doses of MIMP;

FIG. 10 is a bar chart illustrating an adjuvant effect of MIMP on DTH response to Flu challenge in intramuscularly Immunized mice;

FIG. 11 is a chart illustrating antibody titers for mice immunized with Flu via an intramuscular route, wherein the dose of adjuvant is indicated on the X-axis;

FIG. 12 is a bar chart illustrating that an addition of MIMP to influenza vaccine enhances IFN-γ production from splenocytes of aged mice, wherein the splenocytes harvested from aged (20-22 months) C57/Bl/6J mice following 3 intranasal immunizations of 100 μg/mouse influenza peptide epitope vaccine (see Levi, R & R. Arnon, Vaccine 14: 85-92. 1996.)±200 μg/mouse MIMP are cultured with or without inactivated virus (A/Texas/77/1), and supernatants are collected after 70 hours and assayed by ELISA;

FIG. 13 is graph illustrating that MIMP protects mice against listeria monocytogenes infection, wherein MIMP in PBS is administered orally (by gavage) or parenterally starting 5 days prior to infection of male Balb/c mice, 1.7×10⁴ cells of mouse-adapted L. monocytogenes strain EGD (in 0.5 ml PBS) are injected parenterally on day 0, and the control is saline without MIMP (Courtesy of T. Semenenko, unpublished results and U.S. Pat. No. 5,614,504);

FIG. 14 is a graph illustrating that MIMP protects mice against salmonella typhimuirium infection, wherein Balb/c mice are injected intraperitoneally with varying concentrations of MIMP in a fixed volume of PBS at varying times prior to (or post) inoculation with mouse adapted salmonella typhimurium strain 415, a dose of 5×10⁴ bacterial cells in 0.5 ml PBS is administered intraperitoneally at time 0, and the control is saline without MIMP (Courtesy of T, Semenenko, unpublished results and U.S. Pat. No. 5,614,504); and

FIG. 15 is a graph illustrating that MIMP prolongs the survival of mice infected with Friend Leukemia Virus (“FLV”), wherein Balb/c female mice are given a lethal dose (0.2 ml intraperitonally) of FLV virus (−5×10² virus/ml) that results in a mean survival time of 39 days, and groups of 15 mice are treated with either saline or 1 mg/kg MIMP intraperitonally as indicated, starting 3 days after virus inoculation.

### DETAILED DESCRIPTION OF THE INVENTION

Generally, the present invention provides for a pharmaceutical composition used alone, or in conjunction with substances for enhancing immunogenic activity of a subject. Specifically, the pharmaceutical composition includes a protected inosine 5′-monophosphate (“IMP”) compound wherein the protected IMP compound can be used in therapeutic treatment against a pathogen challenge and/or as an effective adjuvant utilized in a vaccine formulation. In any case, the present invention is capable of staying infection and boosting immunity.

As previously discussed in the background section, current vaccines are not always very effective, especially in populations with decreased and/or suppressed immune systems. The present invention provides for novel pharmaceutical compositions that not only increase general immunogenic activity of an individual, but also specifically optimize T-cell responses. Vaccines can take a period of time to produce a therapeutic effect. The present invention, particularly the protected IMP compound, can initially protect infected individuals thereby providing a therapeutic effect until the vaccine induced therapeutic effect arises.

A major roadblock to development of an effective vaccine is the lack an appropriate immune activator (adjuvant) that stimulates both size of the response (T-helper cells) and the cellular immune component of the response (T-cytotoxic cells). All vaccine candidates would benefit from an effective T-cell activator, preferably one that would be independent of the vaccine formulation. Methyl-5′ inosine monophosphate (“MIMP”) is an ideal candidate as an effective T-cell immune stimulator for use with vaccines.

In order to treat bacterial pathogens or viruses, the composition of the present invention stimulates cellular or T-cell immunity. Alternatively, the composition also stimulates humoral or B-cell immunity. The composition of the present invention can be enzyme resistant. The composition stimulates immunity through various mechanisms including, but not limited to, stimulating macrophages and dendritic cells, stimulating T-cells and/or B-cells, inhibiting or activating the functionality of enzymes, affecting cell surface receptors, potentiating T-cell and/or B-cell immunity, and other similar immunity stimulation mechanisms known to those of skill in the art. The composition of the present invention can be given before (for prevention) or after (for treatment) the diagnosis of a viral infection or bacterial infection. The components of the composition of the present invention are all given in effective amounts to provide sufficient immune stimulation.

The term “vaccine agent” includes, but is not limited to, proteins, peptides, coat proteins, viral coats, viruses, bacteria, antigen, whole cells, cell components, parasites, pathogens, and any other vaccine agent known to those of skill in the art.

The term “antiviral and antimicrobial agent” as used herein means compounds that inhibit and/or prevent replication of a pathogen.

The terms “adjuvant” and “adjuvants” as used herein are meant to include, but are not limited to, a substance that aids in enhancing antigenicity and providing a superior immune response. Generally, the substance is mixed with an antigen to produce the desired effect.

Examples of adjuvants that can be used with the present invention include, but are not limited to, cytokines, lipopolysaccarides, pluronic polymers, muramyl dipeptide, lipid A, liposomes, nonphospholipid liposomes, proteoliposomes, homo- and copolymers of lactic and glycolic acids, copolymers, lipidated peptides, aliphatic nitrogenous bases, amines, quaternary ammonium compounds, guanidines, benzamidines, thiouroniums, aluminum hydroxide, aluminum salts, mineral oil, killed microbacteria, detergent, immunostimulators, PCPP salt, aluminum phosphate gel, algal glucan, algammulin, alhydrogel, antigen formulation, N—N-dioctadecyl-N¹,N¹-bis(2-hydroxyethyl)propanediamine, BAY R1005, Calcitriol, calcium phosphate gel, cholera holotoxin, cholera toxin B subunit, cholera toxin A1-subunit-Protein, D-fragment fusion protein, CRL 1005, cytokine-containing liposomes, DDA, Dehydroepiandrosterone, DHA, DMPC, DMPG, DOC, alum complex, Fruend's Complete Adjuvant, Freund's Incomplete Adjuvant, Gerbu Adjuvant, GM-CSF, GMDP, Imiquimod, DTP-GDP, immunoliposomes containing antibodies and/or co-stimulatory molecules, Interferon-γ, Interleukin-1β, Interleukins, Interleukin-2, Interleukin-7, Interleukin-12, Immune stimulating complexes, complexes of saponin derivatives, liposomes, loxoribine, LT-OA, MF 59, Montanide ISA Adjuvants, Squalene/water emulsions, MDP, MTP-PE, MTP-PE Liposomes, Murabutide, Murametide, Murapaimitine, D-Murapalmitine, NAGO, Non-Ionic Surfactant Vesicles, Pleuran, PLGA, PGA, PLA, Pluronic L121, PMMA, Proteinoid microspheres, Poly rA, Poly rU, Polysorbate 80, Protein Cochleates, QS-21, Quil-A, Rehydragel HPA, Rehydragel LV, S-28463, SAF-1 Sciavo peptide, Span 85, Arlacel 85, sorbitan trioleate, Specol, Squalane, Stearyt Tyrosine, DTP-DPP, Thereonyl-MDP, Ty Particles, Walter Reed Liposomes, Hunter's TiterMax, Ribi's Adjuvants, Nitrocellulose-Adsorbed Proteins, Encapsulated Antigens, combinations thereof, and any other similar adjuvants known to those of skill in the art. The present invention provides for a protected IMP compound utilized as an adjuvant, and optionally, utilizing an additional adjuvant as described above.

The term “therapeutic” as used herein means a treatment and/or prophylaxis. A therapeutic effect is obtained by suppression, remission, or eradication of a disease state caused by pathogenic means. Thus, a combination of a positive or negative modulator and suppressor can result in a therapeutic effect.

The term “effective amount” as used herein means an amount that is determined by such considerations as are known in the art of treating secondary immunodeficiencies wherein it must be effective to provide measurable relief in treated individuals, such as exhibiting improvements including, but not limited to, improved survival rate, more rapid recovery, improvement or elimination of symptoms, reduction of post infectious complications and, where appropriate, antibody titer or increased titer against the infectious agent, reduction in tumor mass, and other measurements as known to those skilled in the art.

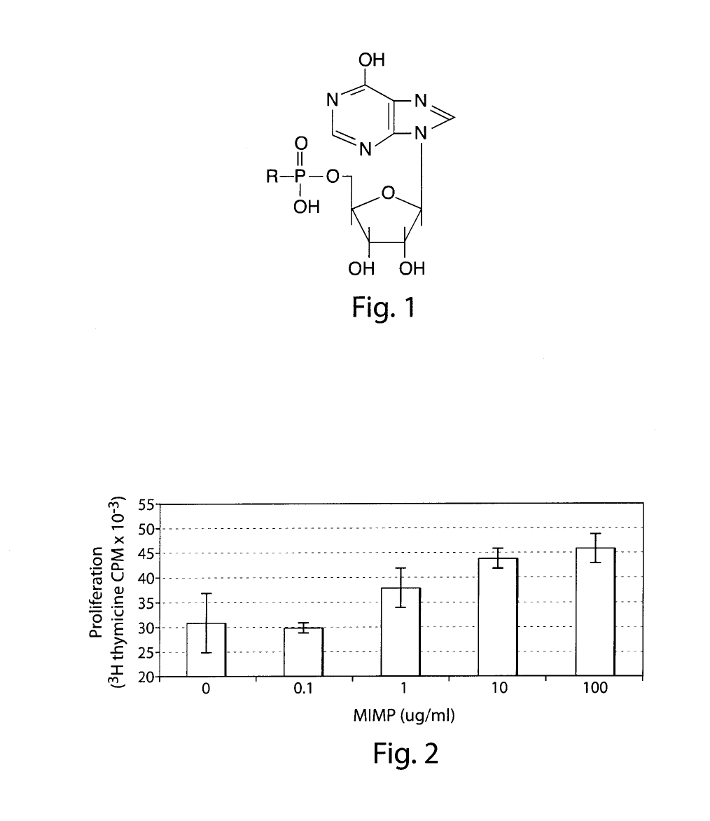

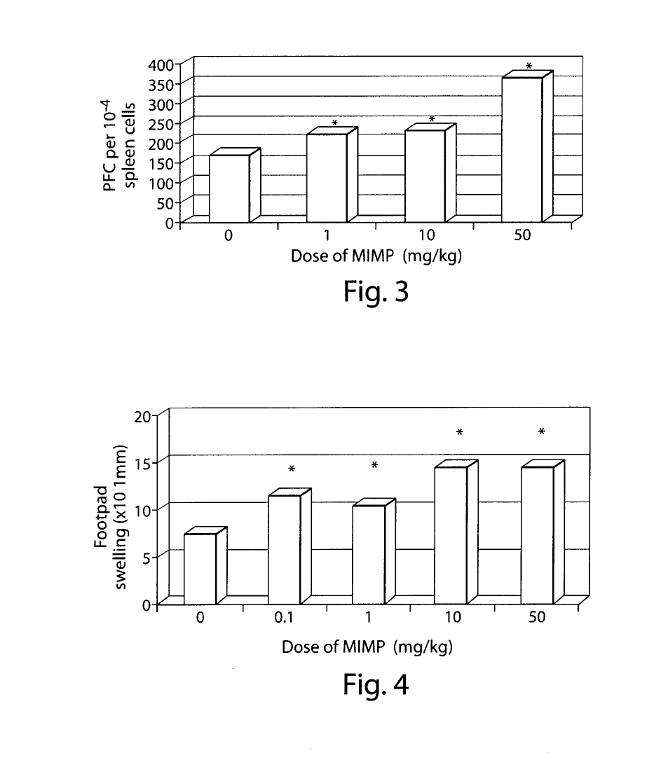

The present invention focuses on the use of an adjuvant effective amount of a protected IMP compound. IMP is an important purine that has great immunopotentiating capabilities. Protected IMP, specifically MIMP, is described in U.S. Pat. No. 5,614,504 to Hadden et al., which is incorporated herein by reference. This immunomodulator is effective in the treatment of infections of intracellular bacterial pathogens and viruses. MIMP has the formula as illustrated in FIG. 1, wherein the R-group is a moiety including, but is not limited to, alkyl, alkoxy, arginine, and secondary amino compounds. The R-group has numerous functions. The R-group can inhibit the hydrolysis of MIMP by 5′-nucleotidase.

MIMP is an inosine-5′-monophosphate derivative. The inosine-5′-monophosphate derivatives are 5′-nucleotidase resistant (“protected-IMP”) that are immunopotentiators. These protected derivatives of inosine-5′-monophosphate as described herein can be readily prepared by condensation of a desired alcohol, primary amine, or peptide with inosine-5′-monophosphate, preferably in the presence of a condensing agent such as dicyclohexylcarbodiimide or the like. Suitable alcohols include monohydric alcohols of 1 to 20 carbon atoms such as methyl alcohol, ethyl alcohol, n-propyl alcohol, n-butyl alcohol, n-hexyl alcohol, n-octyl alcohol, and n-decyl alcohol.

MIMP is active over a broad concentration range to stimulate the responses of murine and human lymphocytes to a T-cell mitogen like PHA and to a more variable degree, B-cell mitogens like LPS and pokeweed. The action of MIMP is further confirmed on responses of enriched human CD4+ and CD8+ lymphocytes. In addition, the results indicate that the suppressive effects of an HIV peptide (See, FIG. 2), IFN-α, and PGE₂ on the PHA response of normal lymphocytes can be reversed if that suppression is mild to moderate and not extreme or toxic. The results also indicate that the lymphocytes of aged and HIV-infected individuals can respond to stimulation by the protected-IMP in the presence of PHA, if the responses are not excessively suppressed.

The preferential action of the 5′-nucleotidase-resistant-inosine-5′-monophosphate on T-lymphocytes demonstrates a receptor-based interaction. MIMP has been shown to induce IL-2 receptors and differentiation markers of prothymocytes (Touraine et al., 1991) in addition to the effects described herein on mature T-lymphocytes.

MIMP, as with any protected-IMP, regulates IL-2 action. IL-2 action has importance in the central role played in orchestrating cellular immune responses mediated by Th1-type T-helper cells. In this regard, it is notable that PHA preferentially elicits a Th1-pattern of cytokines: IL-1, IL-2, g-IFN, and IL-12. It has been shown that PHA does not induce IL-10, IL-3, IL-4, or IL-5. Unpublished data indicate that MIMP has only a small effect on increasing PHA-induced IL-2 or g-IFN, but potently inhibits IL-10 action (See, Example 1). These observations suggest that protected IMP acts on Th1 responses. Specifically, protected IMP has the capacity to inhibit IL-10, which in turn increases Th1 thereof. Hence, protected IMP compounds increase DTH by this mechanism. Since, Th1 responses are critical in the resistance to HIV, cancer, and pathogen infection, e.g., influenza, toxoplasma, listeria, and leishmania (Webster, 2000; Dong et al., 2000; Han and Meydani, 2000; Clerici and Shearer, 1995; Teppler, 1993; Scott and Trinchieri, 1989), the effects of protected IMP compounds to favor these responses support clinical usefulness in such conditions.

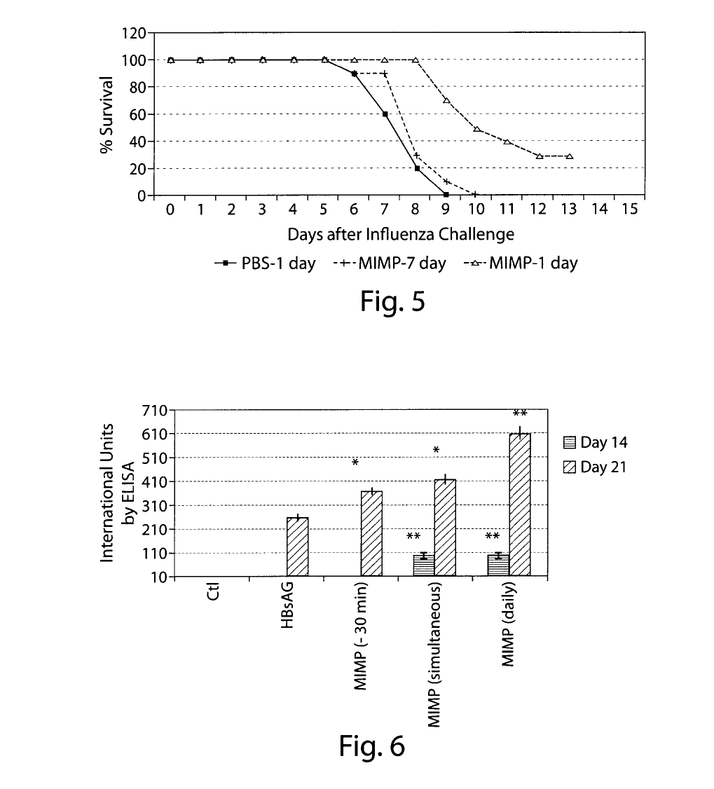

In vivo studies have shown that with protected IMP, DTH is preferentially stimulated over PFC responses and survival has been increased in AIDS, tumor, and infectious challenges (influenza, listeria, salmonella, and FLV; see, FIGS. 5, 13, 14, and 15). In these studies, MIMP proved active by the oral route at doses at or below 1 mg/kg and is nontoxic (oral LD₅₀>2000 mg/kg).

The present invention has numerous embodiments. Generally, the present invention provides for the prevention or treatment of infections of intracellular bacterial pathogens and viruses using an adjuvant effective amount of a protected IMP compound. The present invention provides for a pharmaceutical composition formed by using the protected IMP compound alone, or with additional adjuvants. The combination of the protected IMP compound with additional adjuvants provides for a combination treatment for lethal challenges for which vaccines are available, but may be ineffective against various diseases such as anthrax and small pox.

One embodiment provides for a pharmaceutical composition including an adjuvant effective amount of a protected IMP compound. Additionally, the pharmaceutical composition can include a vaccine agent. The vaccine agent includes, but is not limited to, proteins, peptides, coat proteins, viral coats, viruses, bacteria, antigen, whole cells, cell components, parasites, and pathogens.

The embodiment above can also include additional adjuvants. Examples of these adjuvants have been previously described and listed above.

Another embodiment of the present invention includes an adjuvant effective amount of a protected IMP compound and an effective amount of an antiviral and antimicrobial agent. A further embodiment of the present invention provides for an adjuvant effective amount of an protected IMP compound and an additional adjuvant. Types of adjuvants that can be used with this embodiment can be any adjuvant described above.

Another embodiment of the present invention is for a vaccine formulation including an effective amount of any of the pharmaceutical compositions described herein. The vaccine formulation can be administered alone or be added to existing vaccine formulations known to those of skill in the art. The purpose of the addition of the pharmaceutical composition is for increasing the immune response. Thus, the pharmaceutical composition of the present invention is very useful as an added component in a vaccine composition and/or formulation. The vaccine formulation treats and affects agents including, but not limited to, bacteria, viruses, and any pathogens known to those of skill in the art.

The present invention also provides for vaccine compositions for generating enhanced T-cell immune activity against infectious agents including the pharmaceutical compositions described herein. Further, the vaccine generates enhanced T-cell immune activity against infectious agents including, but not limited to, viruses such as influenza, HIV, hepatitis B, hepatitis C, and smallpox, bacteria such as anthrax, pathogens, and any other infectious agents known to those of skill in the art.

In yet another embodiment of the present invention, there is provided a pharmaceutical composition including an adjuvant effective amount of protected IMP compound described herein and an adjuvant. The adjuvant used can be any adjuvant known to those of skill in the art. Examples of adjuvants that can be used with this pharmaceutical composition have been previously defined herein and described above.

A further embodiment of the present invention is for a pharmaceutical composition including an adjuvant effective amount of the protected IMP compound described above and an anti-HIV substance. Although considerable progress has been made toward controlling HIV infection using combinations of antiviral drugs, elimination of the virus remains a challenge and re-emergence of resistant strains occurs. Although there are a myriad of vaccine candidates, no single candidate has been identified. A major reason for this situation is not that the vaccine candidates are not valid, but that they lack an appropriate immune activator (e.g., adjuvant) that can stimulate both the size of the response (T-helper cells) and the cellular immune component of the response (T-cytotoxic cells).

The anti-HIV substance used with the embodiment of the present invention includes, but is not limited to, HIV proteins, recombinant proteins or peptides, inactivated or attenuated HIV virus, pseudovirus (or other vector) expression of HIV proteins, DNA immunization to product HIV proteins, combinations thereof, and any other similar substances known to those of skill in the art.

Further, one embodiment of the present invention provides for a pharmaceutical composition including an adjuvant effective amount of the protected IMP compound, as described herein, and an antiviral and antimicrobial agent, which has also been described above.

Another embodiment of the present invention is for a pharmaceutical composition including the protected IMP compound as described above and at least one chemotherapeutic substance such as a neurominidase inhibitor. Neurominidase inhibitors are compounds that act as inhibitors of neuraminidase or as intermediates for such inhibitors. The inhibitors bind to locations on the surface or in a cavity of neuraminidase having a geometry unique to neurominidase.

For treatment of the infectious diseases discussed herein, the pharmaceutical composition can be given following exposure to a viral infection or intracellular bacterial pathogen or prior to it. Any of the protected IMP compounds can be included in the pharmaceutical composition. For example, the protected IMP compound is used at a concentration to provide sufficient immune stimulation. The pharmaceutical composition is preferably given within 48 hours of the presence of the symptoms, either daily or at times determined in accordance with good medical practice, taking into account the clinical condition of the individual patient, the site and method of administration, scheduling of administration, and other factors known to medical practitioners. In the case of treating close contacts and family members of an influenza-infected patient, therapy can begin before any symptoms develop or in the case of bioterrorism when an exposure is known.

According to the present invention, the pharmaceutical composition of the present invention is administered in a variety of ways. It should be noted that the pharmaceutical composition can be administered alone or in combination with pharmaceutically acceptable carriers.

The compounds of this invention are formulated with conventional carriers and excipients, which are selected in accord with ordinary practice. Tablets contain excipients, glidants, fillers, binders and the like. Aqueous formulations are prepared in sterile form, and when intended for delivery by routes other than oral administration, generally are isotonic. All formulations optionally contain excipients such as those set forth in the “Handbook of Pharmaceutical Excipients” (1986). Excipients include ascorbic acid and other antioxidants, chelating agents such as EDTA, carbohydrates such as dextrin, hydroxyalkylcellulose, hydroxyalkylmethylcellulose, stearic acid, and the like. The pH of the formulations ranges from about 3 to about 11, but is ordinarily about 7 to about 10.

One or more compounds of the present invention described herein (herein referred to as the active ingredients) are administered by any route appropriate to the condition to be treated. Suitable routes include oral, rectal, nasal, intranasal, topical (including buccal and sublingual), vaginal, parenteral (including subcutaneous, intramuscular, intravenous, intradermal, perilymphatic, intranasal, intraplenic, intrapulmonary, intrathecal and epidural), and the like. It is appreciated that the preferred route can vary with, for example, the condition of the recipient. An advantage of the compounds of the present invention is that they can be administered through a variety of routes.

While it is possible for the active ingredients to be administered alone, it can be preferable to present them as pharmaceutical formulations. The formulations, both for veterinary and for human use, of the invention include at least one active ingredient having at least two pharmaceutical effects or two individual or compounded ingredients, as above defined, together with one or more acceptable carriers, and, optionally, other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations can conveniently be presented in unit dosage form and can be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier, which constitutes one or more accessory ingredients. In general, the formulations are prepared by uniformly and intimately bringing into association the active ingredient with liquid carriers or finely divided solid carriers or both, and then, if necessary, shaping the product.

Formulations of the invention suitable for oral administration are prepared as discrete units such as capsules, cachets, or tablets each containing a predetermined amount of the active ingredient; as a powder or granules; as solution or a suspension in an aqueous liquid or a nonaqueous liquid; or as an oil-in-water liquid emulsion or a water-in-oil liquid emulsion. The active ingredient can be also presented as a bolus, electuary, or paste.

A tablet is made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, surface active or dispersing agent. Molded tablets can be made by molding in a suitable machine a mixture of the powdered active ingredient moistened with an inert liquid diluent. The tablets can be optionally coated or scored and optionally are formulated so as to provide slow or controlled release of the active ingredient therefrom. In one embodiment, acid hydrolysis of the medicament is obviated by use of an enteric coating.

For infections of the eye or other external tissues, e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w, and most preferably, 0.5 to 10% w/w. When formulated in an ointment, the active ingredients can be employed with either a paraffinic or a water-miscible ointment base. Alternatively, the active ingredients can be formulated in a cream with an oil-in-water cream base.

If desired, the aqueous phase of the cream base can include, for example, at least 30% w/w of a polyhydric alcohol, i.e., an alcohol having two or more hydroxyl groups such as propylene glycol, butane 1,3-diol, mannitol, sorbitol, glycerol, polyethylene glycol (including PEG 400), and mixtures thereof. The topical formulations can desirably include a compound, which enhances absorption or penetration of the active ingredient through the skin or other affected areas. Examples of such dermal penetration enhancers include dimethyl sulphoxide and related analogs.

The oily phase of the emulsions of this invention can be constituted from known Ingredients in a known manner. While the phase can comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic. emulsifier, which acts as a stabilizer. It is also preferred to include both an oil and a fat. Together, the emulsifier(s) with or without stabilizer(s) make up the so-called emulsifying wax and the wax together with the oil and fat make up the so-called emulsifying ointment base that forms the oily dispersed phase of the cream formulations.

Emulients and emulsion stabilizers suitable for use in the formulation of the Invention include Tween® 60, Span® 80, cetostearyl alcohol, benzyl alcohol, myristyl alcohol, glyceryl mono-stearate, and sodium lauryl sulfate.

The choice of suitable oils or fats for the formulation is based on achieving the desired cosmetic properties. The cream should be preferably a non-greasy, non-staining, and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate, or a blend of branched chain esters known as Crodamol CAP can be used. These can be used alone or in combination depending on the properties required. Alternatively, high melting point lipids such as white soft paraffin and/or liquid paraffin or other mineral oils are used.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10%, particularly about 1.5% w/w.

Formulations suitable for topical administration in the mouth include lozenges including the active ingredient in a flavored basis, usually sucrose and acacia or tragacanth; pastilles including the active ingredient in an inert basis such as gelatin and glycerin, or sucrose and acacia; and mouthwashes including the active Ingredient in a suitable liquid carrier.

Formulations for rectal administration can be presented as a suppository with a suitable base including, for example, cocoa butter or a salicylate.

Formulations suitable for intrapulmonary or nasal administration have a particle size, for example, in the range of 0.1 to 500 microns (including particle sizes in a range between 0.1 and 500 microns in incremental microns such as 0.5, 1, 30, 35, etc.), which is administered by rapid inhalation through the nasal passage or by inhalation through the mouth so as to reach the alveolar sacs. Suitable formulations include aqueous or oily solutions of the active ingredient. Squalene is a preferred carrier. Formulations suitable for aerosol or dry powder administration can be prepared according to conventional methods and can be delivered with other therapeutic agents such as compounds heretofore used in the treatment or prophylaxis of influenza A or B infections as described below.

Formulations suitable for parenteral administration include aqueous and nonaqueous sterile injection solutions that can contain antioxidants, buffers, bacteriostats, and solutes, which render the formulation isotonic with the blood of the intended recipient, and aqueous and nonaqueous sterile suspensions that can include suspending agents and thickening agents.

The formulations are presented in unit-dose or multi-dose containers, for example, sealed ampoules and vials, and can be stored in a freeze-dried (lyophilized) condition requiring only the addition of the sterile liquid carrier, for example, water for injection, immediately prior to use. Extemporaneous injection solutions and suspensions are prepared from sterile powders, granules, and tablets of the kind previously described. Preferred unit dosage formulations are those containing a daily dose or daily sub-dose, as recited herein, or an appropriate fraction thereof, of the active ingredient.

It should be understood that in addition to the ingredients particularly mentioned above, the formulations of this invention can include other agents conventional in the art having regard to the type of formulation in question. For example, those suitable for oral administration can include flavoring agents.

The invention further provides veterinary compositions comprising at least one active ingredient as above defined together with a veterinary carrier therefor.

Veterinary carriers are materials useful for the purpose of administering the composition and can be solid, liquid or gaseous materials, which are otherwise inert or acceptable in the veterinary art and are compatible with the active ingredient. These veterinary compositions can be administered orally, parenterally, or by any other desired route.

Compounds of the invention are used to provide controlled release pharmaceutical formulations containing, as an active ingredient, one or more compounds of the invention (“controlled release formulations”) in which the release of the compound is controlled and regulated to allow less frequent dosing or to improve the pharmacokinetic or toxicity profile of the compound.

The effective dose of the compound depends at least on the nature of the condition being treated, toxicity, whether the compound is being used prophylactically (lower doses) or against an active influenza infection, the method of delivery, and the pharmaceutical formulation, and is to be determined by the clinician using conventional dose escalation studies. It can be expected to be from about 0.0001 to about 100 mg/kg body weight per day; and typically, from about 0.01 to about 10 mg/kg body weight per day, usually from about 0.01 to about 5 mg/kg body weight per day, and more typically, from about 0.05 to about 0.5 mg/kg body weight per day. For example, the daily candidate dose for an adult human of approximately 70 kg body weight ranges from 0.1 mg to 1000 mg, preferably between 11 mg and 500 mg, and can take the form of single or multiple doses.

Therapeutic compounds of the invention are also used in combination with other active ingredients. Such combinations are selected based on the condition to be treated, cross-reactivities of ingredients, and pharmacological properties of the combination. For example, when treating anthrax exposure, the compound could be combined with an anthrax antigen/vaccine (with or without an antibiotic) to provide for a lowering of the infection, immediate protection, and broader, longer lasting immune mediated protection. Ordinarily, antibiotics, antipyretics, and analgesics are administered together with, or in the same course of, therapy with the compounds of this invention.

Treatment is preferably commenced before or at the time of infection and continued until infection is no longer present. However, the compounds are also effective when given post-infection, for example, after the appearance of established symptoms.

Suitable treatment is given one to four times daily and continued for three to seven days, e.g., five days post-infection depending upon the particular compound used.

While it is possible that, for use in therapy, a compound of the invention can be administered as the raw chemical, it is preferable to present the active ingredient as a pharmaceutical formulation.

The individual components of the combination can be administered either sequentially or simultaneously in separate or combined pharmaceutical formulations.

A pharmaceutical formulation of the present invention can be administered to the patient in an injectable formulation containing any compatible carrier, such as various vehicles, adjuvants, additives, and diluents; or the compounds utilized in the present invention can be administered parenterally to the patient in the form of slow-release subcutaneous implants or targeted delivery systems such as polymer matrices, liposomes, and microspheres. An implant suitable for use in the present invention can take the form of a pellet, which slowly dissolves after being implanted, or a biocompatible delivery module well known to those skilled in the art. Such well-known dosage forms and modules are designed such that the active ingredients are slowly released over a period of several days to several weeks.

Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are well known to those skilled in the art.

The present invention also provides for various methods utilizing the above-described pharmaceutical compositions and/or vaccines. Specifically, there is provided a method of treating influenza by administering an effective amount of the pharmaceutical composition described above. Further, the present invention provides for a method of treating an HIV infection by administering an effective amount of the pharmaceutical composition described herein. Additionally, the present invention provides for a method of treating an infection by administering an effective amount of the pharmaceutical composition described herein. The present invention also provides for a method of enhancing immune resistance to infectious agents by administering an effective amount of an adjuvant effective amount of an protected IMP compound.

In yet another embodiment of the present invention, there is provided a method of enhancing immune response resistance to infectious agents by increasing T-cell activity through the administration of an effective amount of the pharmaceutical composition described herein. Further, this method can optionally include the step of adding an antiviral and microbial agent to yield a more effective clinical response.

The present invention has numerous uses and applications. The present invention is well-suited for either preventive or therapeutic treatments. The composition of the present invention is either administered alone, or with other currently existing vaccines for diseases such as HIV, influenza, hepatitis B, smallpox, anthrax, etc. Thus, in another embodiment, the present invention provides for a method of treating an infection in a subject by increasing T-cell activity and potentiating an immune response by administering an adjuvant effective amount of the protected IMP compound described above.

The composition of the present invention activates T-cell immunity to vaccines as demonstrated in the attached examples. The composition of the present invention is either dependently or independently effective with the vaccines. Also, the composition of the present invention can be administered through numerous routes, as described above.

Additionally, the present invention is useful in the treatment of infections caused by bacteria or viruses. Thus, in an embodiment of the present invention, there is provided a method of affecting an immune response to a viral and microbial agent by potentiating an immune response by administration of an effective amount of the pharmaceutical composition described above. Intracellular bacterial pathogens, which can be prevented or treated using the pharmaceutical of the present invention, include, but are not limited to, tuberculosis, leprosy, Salmonella, Legionella, Listeria and Brucella. Salmonella species are members of the Enterobacteriaceae. Treatment of viral pathogens contemplated by the present invention include, but are not limited to, influenza, hepatitis, herpes, smallpox, and HIV.

In early HIV infection, T-lymphocyte responses have been considered essential to preventing progression to AIDS. T-lymphocyte responses are suppressed by products of HIV including gp16O, gp41, and TAT (see, Good et al., 1991, for review). Recent work suggests that a retroviral peptide, CKS-17, associated with P-15E, can trigger the Th1 to Th2 shift by inhibiting IL-2 and g-IFN production and promoting IL-10 production (Haraguchi et al., 1995). Protected IMP compounds also have been found to augment PHA responses of HIV-Infected individuals. These results indicate that protected IMP compounds, such as those of the present invention, can be employed with antiviral therapy to inhibit progression of HIV-Infected patients to AIDS.

The use of protected IMP compounds in other viral infections is also disclosed by the present invention since immunosuppression attends viral infections of all types studied (Rouse and Horohov, 1988). Immunosuppression accounts for high mortality of influenza in the elderly and high morbidity due to secondary bacterial infections. Interferon production represents one mechanism by which viruses can suppress immunity. The effect of protected IMP compounds to reverse the suppression of IFN-α on the PHA response recommends its application in this regard.

As previously described, the present invention is useful in treating various individuals, particularly the elderly, those infected with anthrax, and any immunocompromised individual. Thus, the present invention provides for methods of treating elderly individuals suffering from an infection by administering an effective amount of the pharmaceutical composition described herein. This method can also include the step of administering an adjuvant effective amount of a protected IMP compound. Moreover, there is provided a method of treating individuals infected by anthrax by administering an effective amount of the pharmaceutical composition described herein.

The above discussion provides a factual basis for the utility of the composition, vaccine, and methods associated with the present invention. The methods used with and the utility of the present invention can be shown by the following non-limiting examples and accompanying figures.

### EXAMPLES

### Example 1

### MIMP Reversal of Immunosuppression by Pathogens

Pathogens utilize a variety of strategies to circumvent the immune system, often through the active suppression of the normal “anti-pathogen” response. Pathogen induced or produced factors such as IFNα, IL-10, as well as a peptide from HIV gp41 itself are associated with this in vivo immunosuppression. In vitro, these agents, as well as others such as prostaglandins and corticosteroids have been shown to inhibit the mitogen-induced. proliferative responses to human PBMC. IL-10 has a more general role in the downregulation of immune responses in part via blocking activation of cytokine synthesis, especially IFNγ and TNFα, normally associated with a Th1 response and in fact, IL-10 production is associated with Th2 activity.

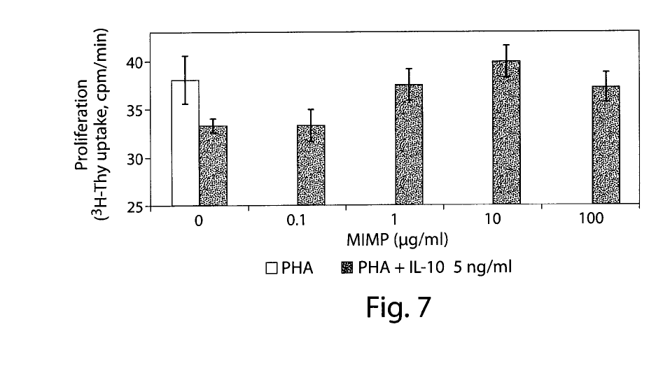

In FIG. 7, it is shown that MIMP can overcome the IL-10 mediated immune suppression of PHA-induced proliferation of human PBMC. Similar results have been demonstrated for MIMP with all of the above-named immunosuppressive agents indicating that MIMP favors a shift to Th1-type responses that would aid in pathogen eradication.

### Example 2

### MIMP Enhancement of T-Cell Immune Response Via Sc Immunization with a Flu Vaccine

Mice are immunized sc in 3 sites with 5 μg of flu vaccine (mono-valent H2N3). PBS is the primary vehicle for injection. For MIMP, concentrations of 1000 μg/mouse, 500 μg/mouse and 100 μg/mouse are administered with the PBS flu. Mice received a booster immunization at 3 weeks and are challenged in the footpad with either flu antigen or vehicle (PBS) 11 days later. Footpad swelling is measured at 24 hours as an assessment of the in vivo activity of MIMP on enhancing T-cell response to flu vaccine. Serum is taken 7 days after the second immunization to assess antibody (B-cell) response.

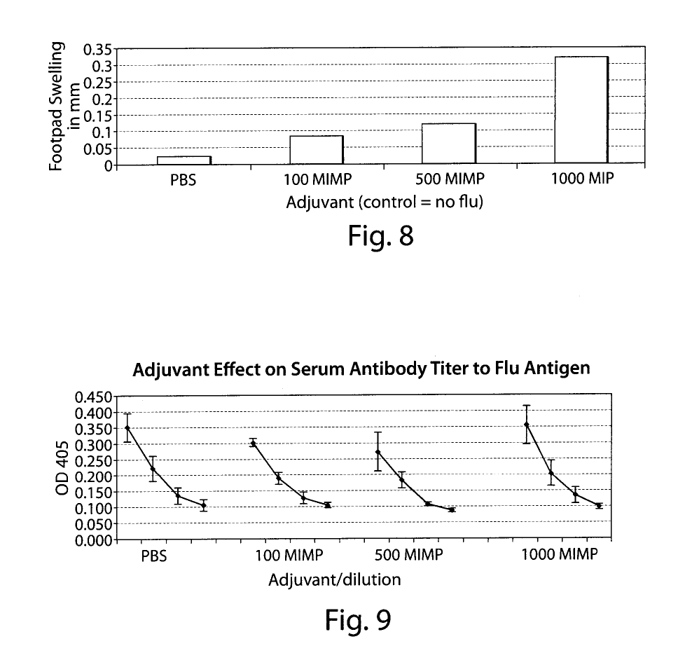

The results of the DTH assay are presented in FIG. 8 as average increase in footpad thickness for the groups of 5 mice. There is no swelling in the mice receiving the PBS-flu immunization. MIMP had a dose-dependent T-cell adjuvant effect.

The antibody data is presented in FIG. 9 as average optical density for the 5 mice per group at each of 4 dilutions. Since the flu antigen is highly antigenic with respect to Initiating a B-cell response, there is no significant increase in titer due to MIMP.

### Example 3

### MIMP Enhancement of T-Cell Immune Response Via an Intramuscular Immunization with a Flu Vaccine

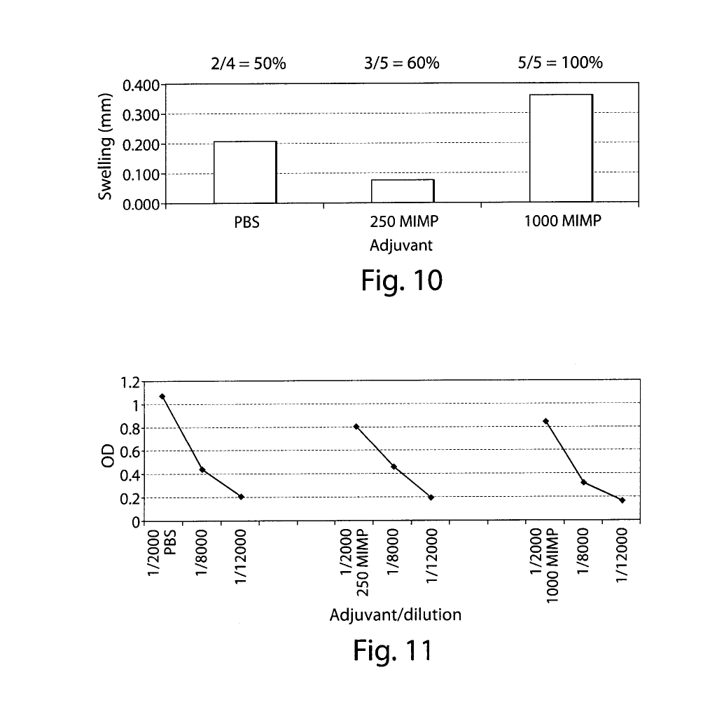

Mice or individuals could be immunized with the flu vaccine and MIMP via an intramuscular route. As shown in FIGS. 10 and 11, mice immunized i.m. with MIMP and flu, mount a T-cell response as defined by a DTH response that is dose-dependent in contrast to the flu vaccine without MIMP (PBS). As with sc immunization, the strong B-cell mediated antibody response to the flu antigen is not significantly influenced by MIMP.

### Example 4

### MIMP-Mediated Enhancement of Th1 Responses in Response to Influenza Vaccination in Aged Mice

Viral infections and intracellular bacterial pathogens are a major health concern. But the precise elements or type of immunity that would be protective are not precisely defined for many diseases. Antibody responses are clearly of benefit, but it is clear that obligate intracellular pathogens, both viral and bacterial, can be only effectively eliminated through the induction of CMI. This is particularly true of viral agents that establish chronic infections, maintaining reservoirs of infected cells, e.g., HIV, HCV, and HSV. Evidence also suggests that complete clearance of influenza virus requires CMI since nude mice, lacking T-cells, chronically shed virus. The experimental results described below indicate that MIMP promotes a protective CMI response following exposure to viral and bacterial pathogens, and that inclusion of MIMP with vaccine antigens can stimulate a cell-mediated response as well as a humoral response.

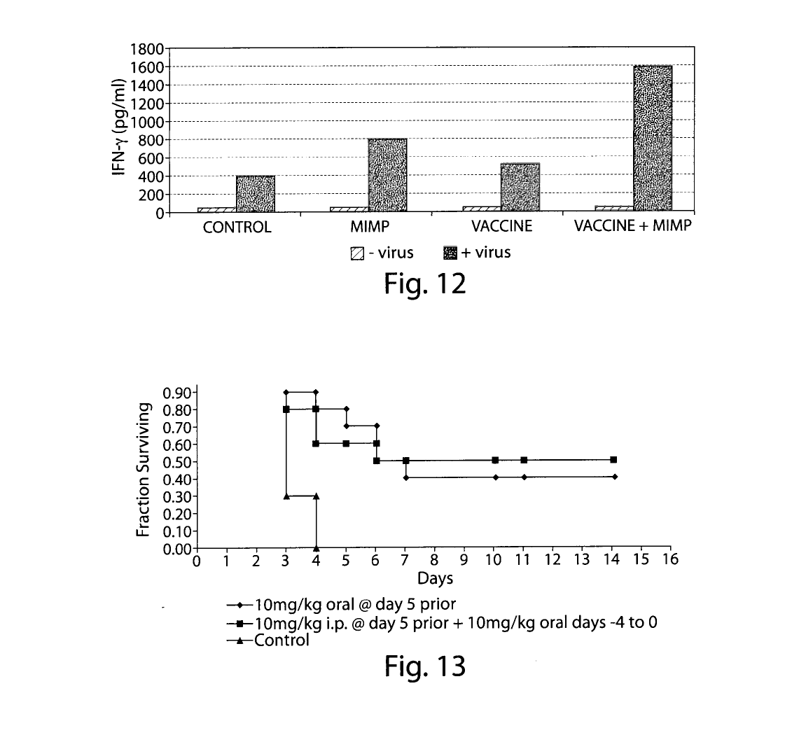

Following immunization with an influenza vaccine, splenocytes are evaluated in vitro for cell-mediated responses to whole virus. The influenza vaccine includes 3 separate influenza A peptides having a B-cell epitope, a CTL epitope and a Th epitope expressed as a hybrid protein. with Salmonella flagellin (Levi, R & R. Arnon, Vaccine 14: 85-92. 1996). Following 3 successive intranasal immunizations (3 week intervals), it is observed that addition of MIMP to the vaccine enhanced in vitro IFN-α production (FIG. 12) from the aged mice in response to whole virus. IFN-α production is not enhanced in similar assays using cells from young (8-10 week old) mice (data not shown), suggesting that the immunological responses to vaccine is already optimal in young mice. While IFN-α is produced, neither IL-4 nor IL-5 is detected in culture fluids from the stimulated splenocytes, further suggesting a preferential Th1 response.

### Example 5

### Orally Administered MIMP Confers Pre-Exposure Protection Against the Intracellular Pathogen Listeria monocytogenes

MIMP is tested in two lethal challenge mouse models with intracellular bacterial pathogens, Listeria monocytogenes and Salmonella typhimurium. Control animals are given a dose of bacteria that caused rapid death with a mean survival time of 2.5 days and 100% mortality by day 4 or 5. As shown in FIG. 13, animals given MIMP intraperitoneally or in a combination of parenteral and oral administration starting 5 days prior to infection have an increased mean survival time (“MST”), and pre-treatment fully protected 40-50% of the animals challenged with Listeria.

### Example 6

### MIMP Pre-Treatment Confers Nonspecific Protection to Salmonella Challenge

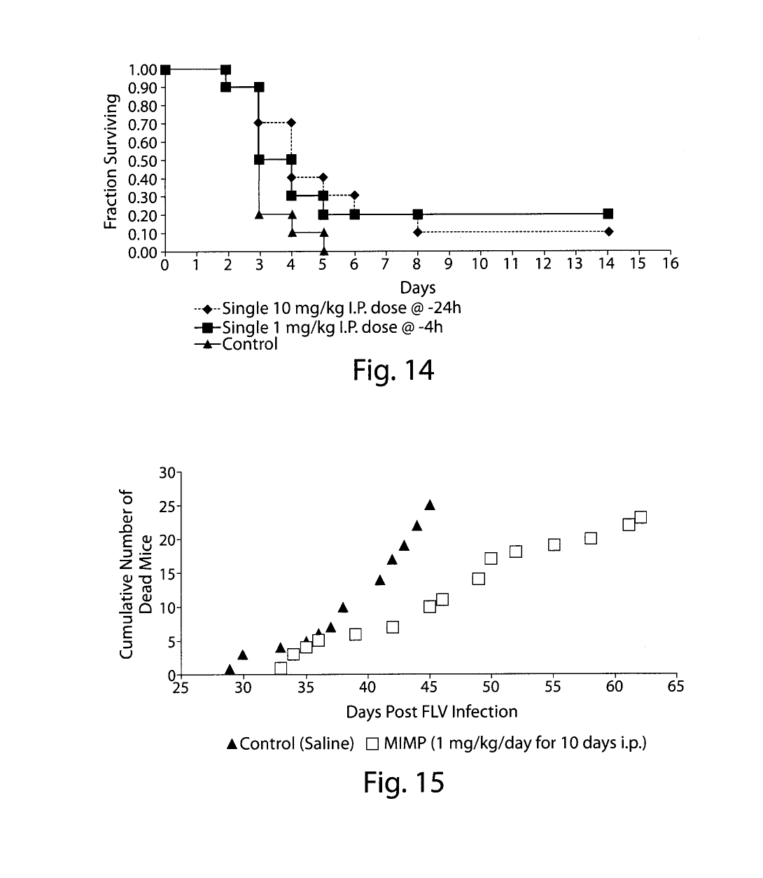

In the Salmonella lethal challenge model, shown in FIG. 14, parenteral dosing of MIMP from 24 to 4 hours prior to infection also resulted in prolongation of MST and protection of 10-20% of treated mice. A similar level of protection (10%) and MST extension is also seen when MIMP is administered at 4 hours post-inoculation across the entire dose range from 0.1 mg/kg to 10 mg/kg.

### Example 7

### Post-Exposure Protection Against Viral Challenges

MIMP also demonstrated post-exposure protective activity against two different viral challenges: FLV, as shown below, and influenza. Infection of mice with FLV (Friend Leukemia Virus) is rapidly lethal with a MST of 39 days. In a stringent test of efficacy, initiating treatment at day 3 after infection, parenteral administration of MIMP (1 mg/kg/day) for 10 days yielded a 26% Increase in MST to 46 days as shown in FIG. 15.

### Example 8

### MIMP Enhancement of T-Cell Immune Response to HIV Vaccine

Although considerable progress has been made towards controlling HIV Infection using a combination of antiviral drugs, elimination of the virus remains a challenge and re-emergence of resistant strains occurs. Thus, development of an HIV vaccine with preventive or even therapeutic activity remains a high priority of research. Although there are a myriad of vaccine candidates, no single candidate has been identified. In this example, applicants demonstrate that orally administered MIMP is an effective T-cell immune stimulator for candidate HIV vaccines. The demonstration that such a compound, especially one that has potential for oral administration, would be of significant commercial value because it could be used in a variety of settings where vaccines are not performing as desired.

A vaccine formulation that is peptide in nature (as an example) offers the benefit of ease of manipulation in the animal model, known strain specificity of the response, and the known weakness of the method, namely narrow and weak humoral and cellular responses. Enhancement of a synthetic peptide based vaccine serves as an example of the efficacy for MIMP to enhance T-cell immune responses to HIV vaccines. Both the ip and oral routes for MIMP can be used and it is expected that the oral route will require 10 fold more material than the ip route.

MIMP (ip or oral) is given at doses between 1 μg and 500 μg with the HIV-peptide with or without ovalbumin conjugation at 100 μg/mouse. Each treatment group contains 10 mice. The DTH assay assesses the response to ovalbumin as a carrier and/or the peptide. Spleen cells are used to assess the T-cell responses in vitro by ELISPOT. Serum is used to assess the antibody response.

### Example 9

### Combination Efficacy of MIMP with Respect to a Protective Response to Infection and Enhancement of T-Cell Immune Response with a Vaccine

Considerable research effort is currently being focused on the issue of combating bioterrorism. A central concern is that agents such as anthrax or smallpox could be released into a general population that is not previously vaccinated against the agent. It is useful if a formulation of the vaccine also contained the capacity to enhance immediate immune activity against the invading organism. Such activation would reduce the burden and provide for an opportunity for the vaccine to take effect. As envisioned in this example, MIMP is formulated with the vaccine and would provide immediate protection as demonstrated in examples 4, 5, and 6 as well as long-term immune activation via the vaccine component of the formulation.

Throughout this application, various publications, patents, and published applications are referenced by author and year, patent number, or application number. Full citations for the publications are listed below. The disclosures of these publications, patents, and published applications in their entireties are hereby incorporated by reference into this application in order to describe the state of the art to which this invention pertains.

The invention has been described in an illustrative manner, and it is to be understood that the terminology, which has been used, is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

### REFERENCES

Altromin 1314, Altromin, Lags, Germany
Bamford, M. J., “J. Enzyme Inhibition” 10:1-6 (1995).
Cierici and Shearer, 1995.
Dong et al., 2000.
Fundamental Virology” (Raven Press, New York, 1986), Chapter 24.
Glasky, 1985
Good et al., 1991
Han and Meydani, 2000.
Hadden et al. 1983.
Hadden, 1987.
Hadden, “T-Cell Adjuvants,” Int. J. Immunopharmac., Vol. 16, No. 9, pp. 703-710 (1994).
Haraguchi et al., 1995.
Masihi, 2000; 1999; 1994
Mills, Stites and Terr, pgs 646-656.
Poland and Couch, 1999
Remington's Pharmaceutical Sciences (Mack Publishing Co., Easton, Pa.).
Rouse and Horohov, 1986.
Saha, et al. 1988.
Scott and Trinchieri, 1989.
Sosa et al., 1992.
Stites and Terr, pgs 637-645
Teppler, 1993.
Touraine et al., 1991.
Webster, 2000.

### U.S. Patents

U.S. Pat. No. 3,728,450 to Gordon.
U.S. Pat. No. 4,221,794 to Hadden et al.
U.S. Pat. No. 4,221,909 to Hadden et al.
U.S. Pat. No. 4,340,726 to Giner-Sorolla et al.
U.S. Pat. No. 4,221,910 to Giner-Sorolla et al.
U.S. Pat. No. 4,457,919 to Giner-Sorolla et al.
U.S. Pat. Nos. 4,510,144 and 4,387,226 to Giner-Sorolla et al.
U.S. Pat. No. 4,487,603.
U.S. Pat. No. 4,486,194.
U.S. Pat. No. 4,447,233.
U.S. Pat. No. 4,447,224.
U.S. Pat. No. 4,439,196
U.S. Pat. No. 4,475,196
U.S. Pat. No. 5,614,504

### Other Patents

WO 91/16320, WO 92/06691.

1-36. (canceled)

37. A method for protection of a subject against an infectious agent, the method comprising:
administering prophylactically an effective amount of a protected inosine-5′-monophosphate (IMP) compound to the subject, and
administering prophylactically an effective amount of a vaccine agent to the subject, the vaccine agent selected from the group consisting of proteins, peptides, viral coats, viruses, bacteria, antigen, whole cells, cell components, parasites, and pathogens,
wherein the method generates in the subject enhanced T-cell immune activity against the infectious agent.

38. The method of claim 37, wherein the protected IMP is a methyl-5′-inosine monophosphate (MIMP).

39. The method of claim 38, wherein the MIMP is of the formula: 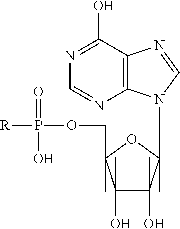
wherein said R-group is a moiety selected from the group consisting of alkyl, alkoxy, arginine, and secondary amino compounds.

40. The method of claim 37, wherein the vaccine agent is a peptide.

41. The method of claim 37, wherein the infectious agent is a human immunodeficiency virus (HIV), a flu virus, or an anthrax bacterium.

42. The method of claim 41, wherein the infectious agent is a flu virus.

43. The method of claim 37, wherein the administration is subcutaneous, intramuscular, or intranasal.

44. The method of claim 40, wherein the administration is subcutaneous, intramuscular, or intranasal.
